(19) United States
(12) Patent Application Publication
(10) Pub. No.: US 2025/0213468 A1
(43) Pub. Date: Jul. 3, 2025

(54) AN ORGANIC CHEWABLE COMPOSITION COMPRISING CERTIFIED ORGANIC INGREDIENTS, AND PROCESS FOR PREPARING THE SAME

(71) Applicant: PharmaChem Laboratories, LLC, Kearny, NJ (US)

(72) Inventors: Kapish KARAN, Wilmington, DE (US); Mahimnsinh PARMAR, Budd Lake, NJ (US); Junior Jean PAUL, Wilmington, DE (US)

(73) Assignee: PharmaChem Laboratories, LLC, Kearny, NJ (US)

(21) Appl. No.: 18/714,021

(22) PCT Filed: Nov. 29, 2022
(86) PCT No.: PCT/US2022/051228

§ 371 Date: May 28, 2024

Related U.S. Application Data
(60) Provisional application No. 63/285,602, filed on Dec. 3, 2021.

(51) Int. Cl. A61K 9/00; A61K 31/375; A61K 31/593; A61K 31/714; A61K 35/00; A61K 35/745; A61K 35/747; A61K 36/064; A61K 36/185; A61K 36/286; A61K 36/899; A61K 47/36; A61K 47/44; A61K 47/46

(52) U.S. Cl. CPC A61K 9/0056; A61K 31/375; A61K 31/593; A61K 31/714; A61K 35/745; A61K 35/747; A61K 36/064; A61K 36/185; A61K 36/286; A61K 36/899; A61K 47/36; A61K 47/44; A61K 47/46; A61K 2035/115

(57) ABSTRACT

Disclosed herein is an organic chewable composition comprising: (i) 0.1 to 95 wt. % of an organic chewable base composition comprising: (a) 70 to 90.0 wt. % of at least one agglomerated organic primary sweetener blend; (b) 1.0 to 10.0 wt. % of at least one organic lubricant and/or an organic binding agent; (c) 1.0 to 10.0 wt. % of at least one co-processed organic secondary sweetener and an organic binding agent; (d) 0.1 to 10.0 wt. % of at least one additive; and (ii) 0.01 to 5 wt. % of at least one functional ingredient. Further disclosed is a process for preparing the organic chewable base composition and a formulation of the chewable base composition into organic chewable vitamin tablets.

The present disclosure relates to an organic chewable composition comprising certified organic ingredients, more particularly, relates to a certified organic chewable composition comprising an organic chewable base composition and at least one functional ingredient.

### BACKGROUND OF THE INVENTION

Chewable products find wide applications in pharmaceutical, nutraceutical and confectionary industries. Chewable products are advantageous in that they are cost effective, require no water for consumption, easily swallowable, flexible in packaging and shipping. Further, these products facilitate more rapid release and more rapid absorption of active ingredients and immediate onset of action. Chewable products are prepared by compressing compounds like active ingredients, flavors, and binders. These products should break when chewed in between the teeth before ingestion and their hardness is directly dependent on pressure applied during compression. Chewable products are prepared from ingredients which are mostly synthetic in origin. When these ingredients are replaced by certified organic products, retention of desired hardness compatible for chewing is challenging. Formulators are looking for an organic chewable base while retaining desired hardness and other physical parameters.

US Patent application US2019/0388353A1 assigned to Genexa Inc. discloses a chewable tablet formulation comprising: an active ingredient, organic dextrose, organic Nu Rice Bran, organic Acai Berry, organic carnauba wax, organic maltodextrin wherein the organic maltodextrin vs active ratio is prepared by trituration prior to tableting in a ratio of about 1:6-13.

US Patent application US2010/0226904A1 assigned to Hero Nutritional Products LLC discloses a chewable composition comprising: an organic compliant binding agent; an organic sweetener; and an active ingredient.

US Patent application US2009/0197974 assigned to Enzymatic Therapy Inc. discloses an all-natural or substantially all-natural dietary supplement in a tablet form that comprises at least 95 percent Certified Organic ingredients by weight and exhibits desirable hardness, friability, wherein release of the active dietary supplement is formulated with naturally occurring saccharides that exhibit tablet binder, tablet disintegrant, or both tablet binder and tablet disintegrant functionality.

Despite these disclosures, there is a need in the art for preparing a commercial organic chewable product, wherein the product has hardness and texture compatible for chewing, more than 95% of the ingredients are organically produced, and the final chewable product is certified “organic”, by certification agencies accredited by Government bodies such as United States Department of Agriculture (USDA).

### SUMMARY OF THE INVENTION

The primary aspect of the present disclosure is directed to an organic chewable composition comprising: (i) 0.1 to 95 wt. % of a chewable base composition comprising: (a) 70 to 90.0 wt. % of at least one agglomerated organic primary sweetener blend; (b) 1.0 to 10.0 wt. % of at least one organic lubricant and/or an organic binding agent; (c) 1.0 to 10.0 wt. % of at least one co-processed organic secondary sweetener and an organic binding agent; (d) 0.1 to 10.0 wt. % of at least one additive; and (ii) 0.01 to 5 wt. % of at least one functional ingredient.

Another aspect of the present disclosure is directed to an organic chewable tablet composition comprising: (i) 0.1 to 95 wt. % of a chewable base composition comprising: (a) 70 to 90.0 wt. % of at least one agglomerated organic primary sweetener blend; (b) 1.0 to 10.0 wt. % of at least one organic lubricant and/or an organic binding agent; (c) 1.0 to 10.0 wt. % of at least one co-processed organic secondary sweetener and an organic binding agent; (d) 0.1 to 10.0 wt. % of at least one additive; and (ii) 0.01 to 5 wt. % of at least one functional ingredient.

In another aspect, the present disclosure is directed to a process for preparing an organic chewable base composition, the process comprising: (a) blending and micronizing 65 to 85 wt. % of at least two organic primary sweeteners using a mill through a 70 mesh to achieve the particle size of 100 to 210 μm; (b) separately agglomerating 1.0 to 10 wt. % of aqueous solution of an organic secondary sweetener and spraying onto the sweetener blend obtained in step (a); (c) separately milling 5.0 to 15.0 wt. % of an organic lubricant and 0.1 to 5 wt. % of an organic binding agent through a 50-mesh to achieve the particle size of 150 to 300 μm and kept aside; (d) separately preparing 5.0 to 15 wt. % of co-processed secondary sweetener and binding agent by mixing a secondary sweetener with an organic binding agent; and (e) mixing the resultant of step (b), step (c) and step (d) along with 0.1 to 10 wt. % of an additive to obtain the organic chewable base.

In another aspect, the present disclosure provides an organic delivery system comprising: (a) a chewable gummy or chewable candy consisting of an organic chewable base composition comprising: (a) 70 to 90.0 wt. % of at least one agglomerated organic primary sweetener blend; (b) 1.0 to 10.0 wt. % of at least one organic lubricant and/or an organic binding agent; (c) 1.0 to 10.0 wt. % of at least one co-processed organic secondary sweetener and an organic binding agent; (d) 0.1 to 10.0 wt. % of at least one additive; and (b) a functional ingredient.

### BRIEF DESCRIPTION OF THE DRAWINGS

In addition to the cited advantages and objects of the disclosure, one or more descriptions of the disclosure briefly summarized can be added by reference to certain embodiments thereof which are illustrated in the appended drawings. These drawings form part of the specification. However, it is to be noted that the appended drawings illustrate preferred embodiments of the disclosure and therefore are not limiting in their scope.

FIG. 1 illustrates Organic Vitamin C tablets with different sweetener blends.

FIG. 2 illustrates Tablet Hardness & Brittleness Test Results of Organic Veg Fruit Fiber tablet (sugar free), Organic Veg-Fruit Fiber tablet (with sugar) and Commercial Veg-Fruit Fiber tablet.

FIG. 3 illustrates Tablet Resiliency Test Results of Organic Veg-Fruit Fiber tablet (sugar free), Organic Veg-Fruit Fiber tablet (with sugar) and Commercial Veg-Fruit Fiber tablet.

FIG. 4 illustrates Bite Toughness Test & Interior hardness test results of organic chewable Vitamin C tablet (sugar free), Organic Chewable Vitamin C tablet (with sugar) and Commercial Chewable Vitamin C tablet.

FIG. 5 is a graph showing the mechanical and dependent parameters for assessing the texture of a tablet.

### DETAILED DESCRIPTION OF THE INVENTION

The technical solutions of the present disclosure will be described clearly and better understood in combination with specific embodiments below, but those skilled in the art will understand that embodiments described below are part of embodiments of the present disclosure but not all of them and are only used for illustration of the present disclosure and should not be considered as limiting the scope of the disclosure. If any specific condition or process are not indicated in the examples, it is to be understood that the conditions are conventionally used by the manufacturer and is commercially available.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by the context, singular terms shall include pluralities and plural terms shall include the singular.

All percentages, parts, proportions, and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

As used herein, the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the term “binding agent” refers to a substance or a mixture of substances added to a tablet to impart a cohesiveness to the tablet formulation which ensures the tablet remaining intact after compression.

As used herein, the term “co-processed” or “co-processing”, refers to a binary mixture of the two components, i.e., sweetener and binding agent, that have been co-processed using various means such as co-milling, mechanical milling, spray drying, freeze-drying, etc.

As used herein, the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “Nutraceutical” refers to a composition comprising a combination of food extracts claimed to have a physiological benefit on human health and/or reduce the risk of chronic disease in humans. Nutraceutical products comprise medicinally or nutritionally functional foods. Nutraceuticals are also called medical foods, designer foods, phytochemicals, functional foods and nutritional supplements. Few examples for nutraceuticals include everyday products as “bio” yoghurts and fortified breakfast cereals, vitamins, herbal remedies and even genetically modified foods and supplements.

As used herein, the term “Organic” or “Certified Organic” or “organically derived” or “organically produced” or “organic origin” or “organically originated” or “natural” or “naturally derived” or “nature derived” or “non-synthetic” are synonymous and interchangeable. Government bodies certify or authorize agencies to certify products as “organic” provided the manufacturers adhere to processes set and approved by these agencies. For ex., in USA, United States Department of Agriculture (USDA) Accredited Certifying Agents for the national Organic Program established in accordance with the Organic Foods Production Act of 1990 (7 U.S.C. 6501 et seq.) certifies organic products. Accordingly, it states—“Organic is a labeling term that indicates that the food or other agricultural product has been produced through approved methods. The USDA organic seal seen on the product label indicates that the product is “certified organic” and has 95 percent or more organic content.

As used herein, the term “Veg” throughout the specification refers to a vegetable fiber used in the preparation of organic chewable formulations.

Present applicants have prepared a commercially viable certified organic chewable base by appropriate selection of certified organic ingredients resulting in a chewable pharmaceutical, nutraceutical or a confectionary product with unique texture and hardness appropriate for comfortable chewing. This is distinguished from the prior art methods, in that, prior arts employ synthetic ingredients.

In one non-limiting embodiment, the present disclosure is directed to an organic chewable composition comprising: (a) 70 to 90.0 wt. % of at least one agglomerated organic primary sweetener blend; (b) 1.0 to 10.0 wt. % of at least one organic lubricant and organic binding agent; (c) 1.0 to 10.0 wt. % of at least one co-processed organic secondary sweetener and an organic binding agent; (d) 0.1 to 10.0 wt. % of at least one additive; and (ii) 0.01 to 5 wt. % of at least one functional ingredient.

Organic chewable composition is produced from ingredients which are certified organic. Certifications assures consumers that product marketed as organic is in fact produced according to organic production standards. Further, certification helps in regulating and expediting the production of organically raised food to consumers. Organic products are certified by agencies from importing countries. For example, In United States, organic farming is regulated domestically by United States Department of Agriculture (USDA) and internationally by Codex Alimentarius guidelines established jointly from the United Nations Food and Agriculture Organization (FAO), the World Health Organization (WHO), and International Federation of Organic Agriculture Movements Certification (IFOAM). To achieve certification in US, fresh fruits and vegetable producers are required to undergo a multistep adoption and recording process that is then inspected by an accredited USDA agent. Only then the farmer's produce is certified “organic”. The Organic Materials Review Institute (OMRI) Certification, and the OMRI web search includes lists and information about materials from the OMRI Generic Materials List© for USDA National Organic Program standards, the OMRI Canada Standards Manual© for Canada Organic Regime standards, and the OMRI Mexico Standards Manual© for the Mexican Organic Products Law standards. In Europe, certification bodies include EU Organic certification, and International Federation of Organic Agriculture Movements Certification (IFOAM) —a German based international NGO. In Australia, the domestic sector can use either the National Association for Sustainable Agriculture standard or the Biological Farmers Association's Australian Organic Standard, while exporters must use an AQIS standard. Further, in India, Global Excellence Certification Inc certifies products as per guidelines of The Agricultural and Processed Food Products Export Development Authority (APEDA), India. Other major certification agencies include California Certified Organic Farmers (CCOF) Certification; and Ecocert Certification. Some countries have consolidated certification at a national level. For example, the United States, the European Union, Canada, and Japan all have organic standards which are formulated and overseen by the government, and the term “organic” may be used only by certified producers.

The present chewable composition is allergan free, xylitol free, and more than 95% of the composition constitutes certified organic ingredients.

In all these agencies, if the products are approved, then the producers are allowed interstate trade under an “organic” label. The present disclosure employs all ingredients which are certified and labelled “organic”. Accordingly, the chewable composition is organic, allergen free, xylitol free, and more than 95% of the composition constitutes certified organic ingredients. Different types of formulation products prepared from the organic chewable base are “Certified Organic” by accredited agencies.

The present organic chewable composition employs sweetening agents or sweeteners, binding agents or binders, lubricants, additives, and functional ingredients.

Primarily, sweeteners or sweetening agents are used to make food more flavorful and to attract consumers. Over time, need for low calories, natural substitutes increased, cutting the use of bulk and intense sweeteners which are mostly synthetic in origin. Consumers are eager to eat products with natural ingredients and clean labels, preferably with other functional properties, and that do not compromise taste. Sweeteners encompass wide-ranging compounds like sugars, sugar alcohols, amino acids, proteins, terpenoid glycosides and some polyphenols.

In a non-limiting embodiment, the sweeteners employed in the present disclosure are organic certified and are categorized into primary and secondary sweeteners. The primary sweeteners impart organoleptic properties to the chewable base.

Non-limiting examples of organic certified primary sweeteners suitable for the present disclosure are selected from the group consisting of Glycyrrhizin or glycyrrhizic acid isolated from Glycyrrhiza glabra L. roots, Thaumatin comprises mixture of six closely related proteins, thaumatin I, II, III, a, b, c, extracted from Thaumatococcus danielli Benth fruit (maraantaceae), Erythritol, fermented product produced using fungi or lactic acid bacteria, Tagatose synthesized using biocatalyst D-galactose into D-tagatose, Steviol glycosides prepared from perennial shrub Stevia rebaudiana (Bertoni) of the Asteraceae family, organic monk fruit extract, organic raw honey, organic blackstrap molasses, organic real maple syrup, organic cane sugar, organic coconut sugar, and mixtures thereof. Natural sweeteners are available commercially in different combinations such as Erysweet+, a stevia erythritol blend, KetoseSweet+, an allulose, stevia and monk fruit blends.

Secondary sweetener is a certified organic sweetener. The secondary sweeteners are dissolved in water to form an aqueous solution. The aqueous solutions of secondary sweetener help in agglomeration of primary sweetener blend during the process for preparing organic chewable base.

Non-limiting examples of secondary sweeteners suitable for the present disclosure is selected from the group consisting of organic isomalto-oligosaccharides, organic chicory root extract, organic maize dextrin, organic rice syrup solids, organic corn maltodextrin, organic tapioca maltodextrin, organic rice maltodextrin, organic potato maltodextrin and blends thereof.

In a non-limiting embodiment, the primary sweeteners are agglomerated blend of organic sweeteners. Granulation process transforms fine powders into free-flowing, dust-free granules that are easy to compress. Agglomeration is a type of granulation process which helps in making more spherical particles mainly using a fluid bed granulation process. Agglomeration imparts many benefits including more uniform blend, improved powder flow, denser, fills more efficiently, lower dusting, less segregation during transit, added functionality, combines multiple ingredients for more efficient handling, and for overcoming sensory issues.

In a non-limiting embodiment, the organic sweeteners are therefore agglomerated to achieve the desired spherical particles having a particle size below 210 μm with D90 in the range of 50 μm to 210 μm using a 70 mesh of US Standard. Further, the agglomerated primary sweeteners blend is mixture of two or more organic sweeteners present in a ratio of 1:10 to 10:1. In another non-limiting embodiment, the other possible ratio includes 1:1, 1:2, 1:3, 1:4, 1:5, 1:6, 1:7, 1:8, 1:9, 1:10, 10:1, 9:1, 8:1, 7:1, 6:1, 5:1, 4:1, 3:1, and 2:1.

In a non-limiting embodiment, the agglomerated primary sweetener blends are present in different combinations including but not limited to (i) Organic Erythritol—Organic Tapioca Maltodextrin blend, (ii) Organic Monk Fruit—Organic Tapioca Maltodextrin blend, (iii) Organic Cane Sugar—Organic Erythritol blend, and (iv) Organic Erythritol—Organic Monk fruit blend.

The agglomerated organic primary sweetener blend is present in an amount of 70 to 90 wt. % of the total chewable base. In another embodiment, the agglomerated organic primary sweetener blend is present in an amount of from about 70 to 75 wt. %; 75 to 80 wt. %; 80 to 85 wt. %; or 85 to 90 wt. % of the total chewable base.

Organic binders or binding agents add cohesiveness to products and provide necessary bonding to form cohesive mass of compact under compression. Binders are conventionally used in direct compression tablets to form compact mass or to mask taste.

In a non-limiting embodiment, certified organic binding agents suitable for use in the present disclosure is selected from the group consisting of polydextrose, dextrins, maltodextrins, indigestible dextrins, gum arabic, fructo oligosaccharides, oligofructose, natural gums, natural gum hydrolysates, gelatin, modified starch and cellulose derivatives. Organic binding agent is present in an amount of at least 1.0 to 10 wt. % of the total chewable base. In another non-limiting embodiment, the binding agent has particle size D90 in the range of 150 μm to 300 μm using a 50 mesh of US standard. In another non-limiting embodiment, the binding agent has particle size D90 in the range of 150 to 200 μm, 200 to 250 μm and 250 to 300 μm using a 50 mesh of US standard. In another non-limiting embodiment, the binding agent has particle size D90 in the range of 200 to 250 μm. In another non-limiting embodiment, the binding agent has particle size D90 is 297 μm. In a non-limiting embodiment, the binding agent is present in an amount of from about 0.1 to 30 wt. %; 0.5 to 20 wt. %; 1 to 20 wt. %; or 1 to 10 wt. % of the total chewable base.

In a non-limiting embodiment, the lubricant is selected from group consisting of an organic palm oil, organic coconut oil, organic stearic acid, organic carnauba wax, organic magnesium stearate and mixtures thereof. In a non-limiting embodiment, the binding agent is present in an amount of from about 0.1 to 30 wt. %; 0.5 to 20 wt. %; 1 to 20 wt. %; or 1 to 10 wt. % of the total chewable base.

In a non-limiting embodiment, the certified organic secondary sweetener and certified organic binding agent is co-processed. Co-processing refers to a binary mixture of the two or more components, i.e., sweetener or blend of sweeteners and binding agent. Co-processing is carried out by co-milling, mechanical milling, spray drying, freeze-drying, etc. The mechanically activated mill can be selected from the group consisting of a roller mill, a ball mill, a bead mill, a Fitz's mill, a millstone mill, a jet mill, and a hammer mill. In another embodiment, the co-processed secondary sweetener and binding agent is present in an amount of from about 0.1 to 30 wt. %; 0.5 to 20 wt. %; 1 to 20 wt. %; or 1 to 10 wt. % of the total chewable base.

In a non-limiting embodiment, the co-processed secondary sweetener-binding agent comprises co-processed tapioca maltodextrin and gum acacia. The ratio of taopica maltodextin to gum acacia is 60 to 80:40 to 20. Tapioca maltodextrin is added to the fluid bed along with half amount of gum acacia while the remaining half amount of gum acacia is mixed with water and is sprayed on to the material in the fluid bed.

In a non-limiting embodiment, the additive is selected from the group consisting of flavoring agents, coloring agents, disintegrants, preservatives, stabilizers, taste enhancing agents, taste masking agents, glidants and lubricants. Additives used in the present disclosure constitutes more than 95% organic certified components.

Non-limiting examples of organic flavors or flavoring agents include oils or extracts having flavors such as apple, acai, acerola, almond, banana, berries, bilberry, blackberry, blueberry, black currant, blessed thistle, botanical flavors derived from cola, boysenberry, beet, cabbage, carrot, coffee, celery, cucumber, cantaloupe, catuaba, cranberry, cherry, cocona, cocoa, chokeberry, citrus fruits, damiana, dandelion, elderberry, green tea, grapefruit, grapeseed, goji berry, ginko, goru kola, hawthorne berry, honeydew, horny goat weed, Japanese knotweed, lemon, lettuce, lime, luo han guo, mango, mandarin, mirabelle, noni, orange, pear, passion fruit, pineapple, peach, plum, pomegranate, papaya, guava, rosehips, rooibos, raspberry, red raspberry leaf, rhubarb, spinach, strawberry, tomato, tea, tangerine, thaumatin, vanilla, vitex berry, watercress, watermelon, white tea, wolfberry, yohimbe and mixtures thereof. In a non-limiting embodiment, the organic flavors are orange, lemon, lime, blueberry, strawberry, mixed berry, cranberry, blackberry, cherry, apple, pineapple, pumpkin, pomegranate acai, and mixtures thereof. The flavoring agent is present in an amount of 0.3 to 5% by weight, based on the total weight of the composition. In a non-limiting embodiment, the flavoring agent is present in an amount of 0.5 to 3% by weight, based on the total weight of the composition.

Organic coloring agents suitable for the present disclosure is selected from the group consisting of organic fruit and vegetable powders like acai, apple, orange, beet, blackberry, bilberry, blueberry, cherry, cucumber, mixed berry, pineapple, pomegranate, pumpkin, strawberry, and mixtures thereof from companies like Naturex, Futureceuticals etc. The organic coloring agent is present in an amount of 0.3 to 5% by weight. In a non-limiting embodiment, the coloring agent is present in an amount of 0.5 to 3% by weight, based on the total weight of the composition.

A lubricant is employed to improve the fluidity of a material. It prevents the formulation from sticking to punches and dies of the tableting machine. In a non-limiting embodiment, lubricants suitable for use in the present disclosure is selected from the group consisting of stearic acid, metallic stearate (e.g., calcium stearate, magnesium stearate, etc.), talc, colloidal silica, sucrose fatty acid ester, hydrogenated vegetable oil, wax, glyceryl fatty acid ester, glycerol dibehenate, rice extract, rice hull powder, gum arabic, sunflower oil and mixtures thereof. Commercially available organic lubricants include Nu-Mag® available as fine powder. The organic lubricant is present in an amount of 0.3 to 5% by weight, based on the total weight of the composition. In a non-limiting embodiment, the coloring agent is present in an amount of 0.5 to 3% by weight, based on the total weight of the composition.

Glidants suitable for the present disclosure is selected from the group consisting of organic Nu-Flow® (organic rice hull powder) marketed by RIBUS, silica marketed by Evonik, etc. The glidant is present in an amount of 0.3 to 5% by weight, based on the total weight of the composition.

Disintegrator or disintegrant is a substance, or a mixture of substances added to a tablet to facilitate its breakup or disintegration after administration. In a non-limiting embodiment, the disintegrant suitable for the present disclosure is selected from the group consisting of sodium starch glycolate, carboxymethyl cellulose calcium, starch, alginic acid, and sodium alginate. The disintegrant is present in an amount of 0.3 to 5% by weight. In a non-limiting embodiment, the disintegrant is present in an amount of 0.5 to 3% by weight, based on the total weight of the composition.

In a non-limiting embodiment, the additive is present in an amount of from about 0.1 to 30 wt. %; 0.5 to 20 wt. %; 1 to 20 wt. %; or 1 to 10 wt. % of the total chewable base.

In a non-limiting embodiment, the functional ingredient is selected from the group comprising a dietary supplement, a mineral, a vitamin, an enzyme, an over-the-counter drug and a pharmaceutical active. Preferably, the functional ingredient is an organic certified chewable dietary supplement. Dietary supplement is selected from the group consisting of riboflavin, pyridoxine, niacin, thiamine, veg-fruit fiber, vitamin C, fermented vitamin B12 and vitamin D, multivitamin and fermented vitamin D.

In another non-limiting embodiment, the functional ingredient is present in an amount of from about 0.05 to 10 wt. %; 0.01 to 5 wt. %; or 0.1 to 5.0 wt. % of the total chewable base.

In another non-limiting embodiment, the Organic certified Chewable base composition is formulated into a tablet, a gummy or a candy.

In another non-limiting embodiment, the present disclosure provides an organic chewable tablet composition comprising: (i) a chewable base composition comprising: (a) 70 to 90.0 wt. % of at least one agglomerated organic primary sweetener blend; (b) 1.0 to 10.0 wt. % of at least one organic lubricant and/or organic binding agent; (c) 1.0 to 10.0 wt. % of at least one co-processed organic secondary sweetener and an organic binding agent; (d) 0.1 to 10.0 wt. % of at least one additive; and (ii) 0.01 to 5 wt. % of at least one functional ingredient.

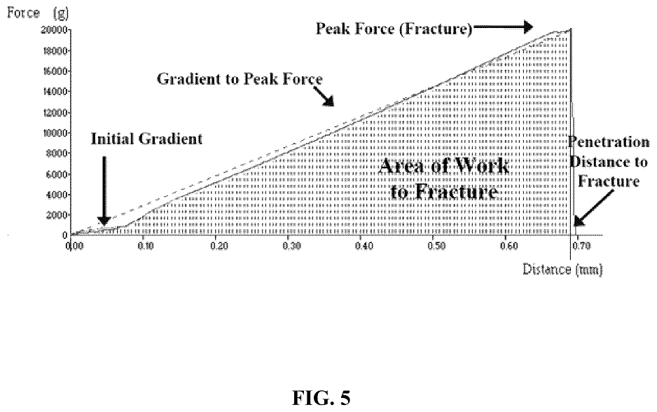

Chewable tablets are one form of delivery systems for pharmaceutical, nutraceutical or confectionary products and their sustenance to varied factors such as production, coating, packaging, and printing makes them more acceptable for oral usage. Several tests are available to assess the strength of the tablet as well as the response to contact with water and other liquids. The texture of any product is multi-faceted and tied to consumer's sensory expectations. Texture Profile Analysis is a widely used double compression test for determining the textural properties of products finding applications in foods, pharmaceuticals, nutraceuticals, and confectionary. Texture Profile Analysis tests are done to determine the mechanical and dependent parameters for assessing the texture of a product. During a TPA test, samples are compressed twice using a texture analyzer to provide insight into how samples behave when chewed. The TPA test was often called the “two bite test” because the texture analyzer mimics the mouth's biting action. An instrument Texturometer was developed by Texture Technologies.com to measure texture of food products based on five basic mechanical parameters (hardness, cohesiveness, adhesiveness, viscosity, and elasticity) and three dependent parameters (brittleness, chewiness, and gumminess). The TA.XT2 Texture Analyzer and Texture Expert Software captures the complete deformation profile of the test. The variability is reduced by testing the center of the tablet, rather than including the areas near the edges which fracture more variably and may be affected by post-compaction sample handling. A ¼″ diameter ball probe is used since it offers a solid contact with the tablet from the moment the tablet is encountered rather than a possibly uneven contact with a flat cylindrical probe. TA. XT texture analysis instrument by Texture Technologies Corp. has the following test specifications (shown in FIG. 5) —
¼″ sphere probe (TA 8) settings:
pretest speed 0.2 mm/s,
test speed 0.2 mm/s,
distance 1.5 mm, and
trigger 20 g force.

The important measures are (i) the force required to fracture the tablet, (ii) the distance at which the fracture occurred (an important indication of brittleness), (iii) the initial gradient (indicating tablet freshness and moisture migration issues), and (iv) the area of work through the fracture point. Distance measures can be independent of fracture force. By profiling the complete deformation through the fracture event, the TA.XT2 captures these differences vis-à-vis disintegrating tablet using conventional instruments.

Hardness value is the peak force that occurs during the first compression. Cohesiveness is how well the product withstands a second deformation relative to its resistance under the first deformation. Measured by the area of work during the second compression divided by the area of work during the first compression. Accordingly, the organic certified chewable tablet of the present disclosure showed an average tablet hardness of about 8 to 30 Kiloponds (kp), or 8 to 20 Kiloponds (Kp). In a non-limiting embodiment, the present chewable composition exhibits chewability without fracture showing high bite toughness with exterior tablet hardness of about 10,000 Kp (kiloponds) and interior tablet hardness of about 1.00 Kp.

Friability is one of the commonly employed test of the ability of tablets to withstand mechanical stresses determines their resistance to chipping and surface abrasion by tumbling them in a rotating cylinder. The percentage weight loss after tumbling is referred to as the friability of the tablets. Fracturability is the measure of the mechanical integrity of tablets is their “breaking force”, which is the force required to produce failure (i.e., breakage) in a specific plane. Not all products fracture; but when they do fracture the Fracturability point occurs where the plot has its first significant peak (where the force falls off) during the probe's first compression of the product. In a non-limiting embodiment, the organic certified chewable tablet of the present disclosure showed friability below 1%.

Resilience is how well a product “fights to regain its original height”. Resilience is measured on the withdrawal of the first penetration, before the waiting period is started. Resilience can be measured with a single compression; however, the withdrawal speed must be the same as the compression speed. Springiness is how well a product physically springs back after it has been deformed during the first compression and has been allowed to wait for the target wait time between strokes. The spring back is measured at the downstroke of the second compression. In some cases, an excessively long wait time will allow a product to spring back more than it might under the conditions being researched (e.g., one would not wait for 60 seconds between chews). Springiness is now expressed as a ratio or percentage of a product's original height. Springiness is measured several ways, but most typically, by the distance of the detected height during the second compression divided by the original compression distance. In a non-limiting embodiment, the organic certified chewable tablet of the present disclosure showed resiliency characterized by lingering resistance for 5 to 15 seconds.

In a non-limiting embodiment, the organic chewable tablet is certified Organic chewable Vitamin C tablet. Non-limiting examples of plant sources for deriving Vitamin C is selected from the group comprising acerola cherry, Indian gooseberry, Elderberry, orange, lemon, and sweet lime.

In a non-limiting embodiment, the functional ingredient is an Organic Veg-Fruit Fiber tablet. The dietary fiber in the fruits and vegetables perform anti-cancer function by removing toxins from the body when consumed on regular basis. Further, vegetable and fruit fibers aid in fat absorption, removal of cholesterol from the body and regulate insulin levels in body. For all these reasons, people of all ages can consume veg fruit fiber and demand for natural veg fruit fiber in tablet or candy forms has been increasing over time. Present inventors have formulated organic certified base composition to form organic certified chewable veg fruit tablet.

Non-limiting examples of organic Veg-Fruit blend include one or more substances derived from the group consisting of organic acerola, organic apple, organic beet, organic blackberry, organic banana, organic cranberry, organic carrot, cucumber powder, organic mango, organic pomegranate juice, organic pineapple, organic raspberry, organic strawberry, organic tart cherry, organic wild blubbery, and organic white rice flour. Commercially available veg-fruit blends include cherry powder derived from fruit of Prunus avium, Blackberry from fruit part of plant Rubus fruticosus, and Cucumber powder from fruit part of Cucumis sativus, all of these are manufactured and marketed by Future Ceuticals.

In a non-limiting embodiment, the functional ingredient is a Fermented Vitamin B12 and Vitamin D derived from natural ingredients selected from the group consisting of Organic Chewable Base and soy free fermented medium. The organic chewable base consists of (Sugar Free) [Organic Sweetener Blend Sugar-Free Blend: (Organic Erythritol, Organic Monk Fruit Powder), Organic Tapioca Maltodextrin, Organic Stearic Acid, Organic rice fiber powder, and Organic Gum Arabic], Organic Natural Flavors, Organic Beet Powder (color). Soy free fermented media consists of Organic Rice Protein Powder, Organic Yeast (Saccharomyces cerevisiae), Organic Tapioca Maltodextrin, Organic Gum Arabic, Organic Black Strap Molasses, Probiotic Blend (Lactobacillus acidophilus, Lactobacillus rhamnoses and Bifidobacterium bifidum), Organic Safflower Oil, Bromelain and Papain.

In a non-limiting embodiment, the functional ingredient is a chewable multi-vitamin tablet derived from natural ingredients selected from the group consisting of Organic Chewable Base and fermented medium. The organic chewable base consists of Organic Sweetener Blend: (Organic Erythritol, Organic Cane Sugar), Organic Tapioca Maltodextrin, Organic Stearic Acid, organic rice fiber powder and Organic Gum Arabic), Organic Natural Flavor, Citric Acid. The fermented medium consists of Organic Rice Protein Powder, Organic Yeast (Saccharomyces cerevisiae), Organic Tapioca Maltodextrin, Organic Gum Arabic, Organic Black Strap Molasses, Probiotic Blend (Lactobacillus acidophilus, Lactobacillus rhamnosus and Bifidobacterium bifidum), Organic Safflower Oil, Bromelain and Papain.

In a non-limiting embodiment, the functional ingredient is a Fermented Vitamin D tablet derived from natural ingredients organic chewable base and soy free fermented medium. The base consists of Organic Sweetener Blend (Organic cane sugar, Organic Erythritol), Organic Tapioca Maltodextrin, Organic Stearic Acid, Organic Ground Rice hulls, Organic Natural Flavors, Organic Beet Powder (color), Organic Gum Arabica and soy free fermented medium consists of Organic Rice Protein Powder, Organic Yeast (Saccharomyces cerevisiae), Organic Tapioca Maltodextrin, Organic Gum Arabic, Organic Black Strap Molasses, Probiotic Blend (Lactobacillus acidophilus, Lactobacillus rhamnoses and Bifidobacterium bifidum), Organic Safflower Oil, Cholecalciferol, Bromelain and Papain.

In another non-limiting embodiment, the present disclosure provides a process for preparing an organic chewable base composition, the process comprising: (a) blending and micronizing 65 to 85 wt. % of at least two organic primary sweeteners using a mill through a 70 mesh to achieve the particles size of 100 to 210 μm; (b) separately agglomerating 1.0 to 10 wt. % of aqueous solution of an organic secondary sweetener and spraying onto the sweetener blend obtained in step (a); (c) separately milling 5.0 to 15.0 wt. % of an organic lubricant and 0.1 to 5 wt. % of organic binding agent through a 50-mesh to achieve the particles size of 150 to 300 μm and kept aside; (d) separately preparing 5.0 to 15 wt. % of co-processed secondary sweetener and binding agent by mixing a secondary sweetener with an organic binding agent; and (e) mixing the resultant of step (b), step (c) and step (d) along with 0.1 to 10 wt. % of an additive to obtain the organic chewable base.

In a non-limiting embodiment, the organic agglomerated primary sweetener blends are selected from (i) Organic Erythritol—Organic Tapioca Maltodextrin blend, (ii) Organic Monk Fruit—Organic Tapioca Maltodextrin blend, (iii) Organic Cane Sugar—Organic Erythritol blend, and (iv) Organic Erythritol—Organic Monk fruit blend.

Accordingly, agglomeration of sweeteners is performed by milling using Hammer mill, jet mill, ball mill, and conical mill.

In a non-limiting embodiment, the co-processed sweetener blend is co-processed tapioca maltodextrin-gum acacia. Accordingly, the co-processed Tapioca Maltodextrin-Gum Acacia is manufactured in a fluid bed process by agglomeration. The ratio of tapioca maltodextrin to gum acacia is 80:20. Tapioca maltodextrin is added to the fluid bed dryer along with half amount of gum acacia while the remaining half amount of gum acacia is mixed with water and is sprayed on to the material in the fluid bed dryer.

In another non-limiting embodiment, the present disclosure provides an organic delivery system comprising: (a) a chewable gummy or a chewable candy consisting of an organic chewable base composition; and (b) a functional ingredient.

In a non-limiting embodiment, the functional ingredient is a dietary supplement, a mineral, a vitamin, an enzyme, an over-the-counter drug or a pharmaceutical active.

Further, certain aspects of the present application are illustrated in detail by way of the following examples. The examples are given herein for illustration of the application and are not intended to be limiting thereof.

### EXAMPLES

The following samples were prepared and subjected to texture profile analysis and sensory evaluation studies.
(a) Organic fruit & veg fiber tablet (sugar free)
(b) Organic fruit & veg fiber tablet
(c) Commercial Veg fiber tablet
(d) Organic chewable Vitamin C tablet (sugar free)
(e) Organic chewable Vitamin C tablet
(f) Commercial Chewable Vitamin C tablet
(g) Fermented Vitamin B12 and Vitamin D Tablet (Certified Organic)
(h) Chewable Multivitamin (Certified Organic)
(i) Fermented Vitamin D Tablet (Certified Organic)

### Example 1: Organic Base Composition (with Sugar)

An organic sugar base was prepared using primary sweetener (Organic Erythritol and Organic Cane Sugar), secondary sweetener (tapioca maltodextrin), binding agent (rice hull powder), lubricant (organic palm fruit stearin) and co-processed sweetener and binding agent (co-processed tapioca maltodextrin and gum acacia). The ingredients along with amounts in percentages are tabulated below.

TABLE 1
Organic Sugar Base

| Chewable Base | % Composition |
|---|---|
| Organic Erythritol | 21.8% |
| Organic cane sugar | 51.4% |
| Tapioca maltodextrin | 5.9% |
| Rice Hulls Powder | 1.4% |
| Co-processed Tapioca Maltodextrin + Gum Acacia | 8.1% |
| Organic Palm Fruit Stearin | 9.5% |
| Silica | 1.8% |
| Total | 100.0% |

### Example 2: Organic Base Composition (Sugar Free)

An organic sugar free base was prepared using primary sweetener (Organic Erythritol and Organic Monk Fruit), secondary sweetener (tapioca maltodextrin), binding agent (rice hull powder), lubricant (organic palm fruit stearin) and co-processed sweetener and binding agent (co-processed tapioca maltodextrin and gum acacia). The ingredients along with amounts in percentages are tabulated below.

TABLE 2
Organic Sugar free Base

| Chewable Base | % Composition |
|---|---|
| Organic Erythritol | 70.8% |
| organic monk fruit | 2.1% |
| Tapioca maltodextrin | 5.9% |
| Rice Hulls Powder | 1.4% |
| Co-processed Tapioca Maltodextrin + Gum Acacia | 8.5% |
| Organic Palm Fruit Stearin | 9.5% |
| Silica | 1.8% |
| Total | 100.0% |

### Example 3: Sweetener Evaluation Dry Blend vs. Agglomerated Sugars

Agglomerated primary sweeteners were blended and tested in comparison with dry blends. Different agglomerated blend vs dry blends compositions are provided below:

### Example 3A: Organic Vitamin C Base (with Sugar)

An organic chewable Vitamin C sugar base was prepared using Organic Acerola Cherry extract, Organic chewable base comprising primary sweetener (Organic Erythritol and Organic Cane sugar), secondary sweetener (tapioca maltodextrin), binding agent (rice hull powder), lubricant (organic palm fruit stearin), co-processed sweetener and binding agent (co-processed tapioca maltodextrin and gum acacia), and orange juice flavor. The ingredients along with amounts in percentages are tabulated below.

TABLE 3
Organic Vitamin C base (with sugar)

| Ingredient | Composition (%) |
|---|---|
| Organic Acerola Cherry Extract | 34.1 |
| Organic Chewable Base (Sugar) | 62.8 |
| Organic orange flavor | 0.8 |
| Organic orange juice flavor | 2.3 |
| Total | 100.0 |

### Example 3B: Organic Vitamin C Base (Sugar Free)

An organic chewable Vitamin C sugar free base was prepared using primary sweetener (Organic Erythritol and Organic Monk Fruit), secondary sweetener (tapioca maltodextrin), binding agent (rice hull powder), lubricant (organic palm fruit stearin) and co-processed sweetener, binding agent (co-processed tapioca maltodextrin and gum acacia) and orange juice flavor. The ingredients along with amounts in percentages are tabulated below.

TABLE 4
Organic Vitamin C base (sugar free)

| Ingredient | Composition (%) |
|---|---|
| Organic Acerola Cherry Extract | 34.1 |
| Organic Chewable Base (Sugar free) | 62.8 |
| Organic orange flavor | 0.8 |
| Organic orange juice flavor | 2.3 |
| Total | 100.0 |

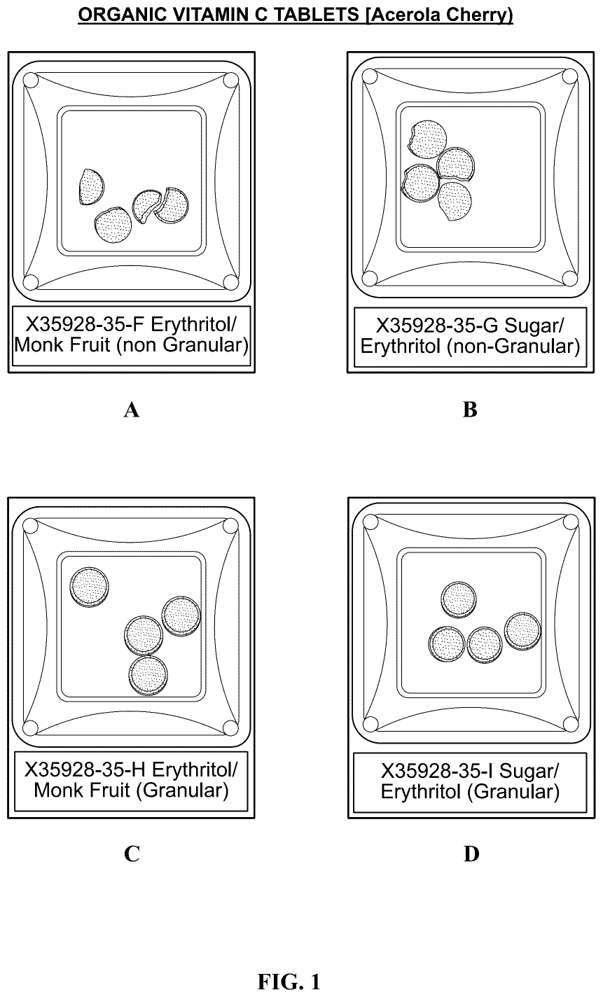

Organic certified chewable Vitamin C tablets using sugar free, and the sugar base manufactured using the current process are provided in FIG. 1. Tablets shown in FIGS. 1C and 1D (bottom two pictures) showed less defects and breakage.

### Example 3C: Organic Chewable Veg-Fruit Fiber Base (Sugar)

An organic Veg-Fruit fiber sugar base was prepared using Organic fruit fiber, Organic vegetable fiber, organic chewable base comprising primary sweetener (Organic Erythritol and Organic Cane sugar), secondary sweetener (tapioca maltodextrin), binding agent (rice hull powder), lubricant (organic palm fruit stearin), co-processed sweetener and binding agent (co-processed tapioca maltodextrin and gum acacia), and orange fruit punch flavor. The ingredients along with amounts in percentages are tabulated below.

TABLE 5
Organic chewable Veg-Fruit fiber base (sugar)

| Ingredient | Composition (%) |
|---|---|
| Organic fruit fiber | 7.69 |
| Organic vegetable fiber | 3.85 |
| Organic Chewable Base (Sugar) | 84.23 |
| Organic fruit punch flavor | 4.23 |
| Total | 100.0 |

### Example 3D: Organic Chewable Veg-Fruit Fiber Base (Sugar Free)

An organic chewable Veg-Fruit fiber sugar free base was prepared using organic fruit fiber, organic vegetable fiber, organic chewable sugar free base comprising primary sweetener (Organic Erythritol and Organic Monk Fruit), secondary sweetener (tapioca maltodextrin), binding agent (rice hull powder), lubricant (organic palm fruit stearin) and co-processed sweetener, binding agent (co-processed tapioca maltodextrin and gum acacia) and orange fruit punch flavor. The ingredients along with amounts in percentages are tabulated below.

TABLE 6
Organic chewable Veg-Fruit fiber base (sugar free)

| Ingredient | Composition (%) |
|---|---|
| Organic fruit fiber | 7.69 |
| Organic vegetable fiber | 3.85 |
| Organic Chewable Base (Sugar free) | 84.23 |
| Organic fruit punch flavor | 4.23 |
| Total | 100.0 |

### Example 4: Fermented Vitamin B12 and Vitamin D Tablet (Certified Organic)

An organic chewable sugar free fermented vitamin B12+vitamin D tablet was prepared using soy free fermented medium comprising Organic Rice Protein Powder, Organic Yeast (Saccharomyces cerevisiae), Organic Tapioca Maltodextrin, Organic Gum Arabic, Organic Black Strap Molasses, Probiotic Blend (Lactobacillus acidophilus, Lactobacillus rhamnoses and Bifidobacterium bifidum), Organic Safflower Oil, Bromelain and Papain. Other ingredients included Organic Chewable Base (Sugar Free) comprising [Organic Sweetener Blend Sugar-Free Blend: (Organic Erythritol, Organic Monk Fruit Powder), Organic Tapioca Maltodextrin, Organic Stearic Acid, Organic rice fiber powder, and Organic Gum Arabic], Organic Natural Flavors, Organic Beet Powder (color). The ingredients along with amounts in percentages are tabulated below.

TABLE 7
Fermented Vitamin B12 and Vitamin D Tablet (Certified Organic)

| Ingredient | Composition (%) |
|---|---|
| Organic chewable base (sugar free) | 83.3 |
| Organic fruit punch FLV | 7.30 |
| Organic Chewable Base (Sugar SOY | 1.60 |
| FREE fermented Vitamin D3 900,000 IU | |
| SOY FREE fermented Vitamin B12 | 0.60 |
| Organic beet color | 7.20 |
| Total | 100.0 |

### Example 5: Chewable Multivitamin

Chewable multivitamin was prepared using organic grains, fruits, and vegetables.

Fermented media consisting of Organic Rice Protein Powder, Organic Yeast (Saccharomyces cerevisiae), Organic Tapioca Maltodextrin, Organic Gum Arabic, Organic Black Strap Molasses, Probiotic Blend (Lactobacillus acidophilus, Lactobacillus rhamnosus and Bifidobacterium bifidum), Organic Safflower Oil, Bromelain and Papain was used. Additional ingredients that were added included Organic Chewable Bae (Organic Sweetener Blend: (Organic Erythritol, Organic Cane Sugar), Organic Tapioca Maltodextrin, Organic Stearic Acid, organic rice fiber powder and Organic Gum Arabic), Organic Natural Flavor, and Citric Acid. The ingredients along with amounts in percentages are tabulated below.

TABLE 8
Chewable multivitamin tablet (Certified Organic)

| Ingredient | % |
|---|---|
| Soy Free Fermented Beta Carotene (50,000 Iu/Gm) | 0.10 |
| Beta carotene 20% | 0.05 |
| Soy free fermented vitamin c 25% | 0.69 |
| Ascorbic acid | 1.00 |
| Soy free fermented vitamin d-3 (900,000 iu/g) | 0.01 |
| VITAMIN D3 500,000 iu | 0.00 |
| Soy free fermented vitamin e natural, (acetate) 250 iu/gm | 0.20 |
| Vitamin e 700 iu | 0.33 |
| Soy free fermented vitamin k-1 (1%) plus gpm | 0.01 |
| Vitamin k1 1% | 0.06 |
| Soy free fermented vitamin b-1 (thiamin) 25% | 0.02 |
| Thiamin hcl 97% | 0.01 |
| Soy free fermented vitamin b-2 (riboflavin) 10% | 0.05 |
| Riboflavin 95% | 0.02 |
| Soy free fermented niacinamide 25% | 0.12 |
| Niacinamine usp | 0.10 |
| Soy free fermented vitamin b-6 (pyridoxine hcl) 20% | 0.03 |
| Pyridoxine hcl 98% | 0.03 |
| Soy free fermented folic acid 1% | 0.16 |
| Folic acid 10% | 0.06 |
| Soy free fermented vitamin b-12 (0.5%) plus gpm | 0.00 |
| Vitamin b12 1.0% | 0.01 |
| Soy free fermented biotin 0.5% | 0.00 |
| Biotin 1% | 0.03 |
| Soy free fermented pantothenic acid | 0.06 |
| D-calcium pantothenate | 0.05 |
| Soy free fermented choline (25.0%) plus | 1.19 |
| Choline bitartrate | 3.21 |
| Soy free fermented calcium (5.0%) plus | 2.72 |
| Calcium carbonate | 10.67 |
| Soy free fermented iodide (1.5%) plus gpm | 0.04 |
| Iodine 5% | 0.04 |
| Soy free fermented magnesium (5.0%) plus | 0.54 |
| Magnesium oxide | 0.33 |
| Soy free fermented zinc (5.0%) plus gpm | 0.31 |
| Zinc oxide | 0.06 |
| Soy free fermented selenium (0.1%) plus gpm | 0.01 |
| Selenium 2.0% | 0.01 |
| Soy free fermented copper (1%) plus gpm | 0.00 |
| Copper sulfate anhydrous | 0.03 |
| Organic chewable base (sugar) | 68.39 |
| Organic fruit punch flavor | 7.26 |
| Organic pineapple flavor | 0.52 |
| Organic lemon juice powder | 1.30 |
| Organic beet powder | 0.26 |
| Total | 100.00 |

### Example 6: Fermented Vitamin D Tablet

Organic chewable vitamin D tablet was prepared using soy free fermented media and additional ingredients. Soy free fermented media consisted of Organic Rice Protein Powder, Organic Yeast (Saccharomyces cerevisiae), Organic Tapioca Maltodextrin, Organic Gum Arabic, Organic Black Strap Molasses, Probiotic Blend (Lactobacillus acidophilus, Lactobacillus rhamnoses and Bifidobacterium bifidum), Organic Safflower Oil, Cholecalciferol, Bromelain and Papain. Additional ingredients Organic Sweetener Blend (Organic cane sugar, Organic Erythritol), Organic Tapioca Maltodextrin, Organic Stearic Acid, Organic Ground Rice hulls, Organic Natural Flavors, Organic Beet Powder (color), and Organic Gum Arabica. The ingredients along with amounts in percentages are tabulated below.

TABLE 9
Fermented Vitamin D Tablet (Certified Organic)

| Ingredient | Composition (%) |
|---|---|
| Organic chewable base (sugar free) | 79.60 |
| Organic fruit punch FLV | 12.00 |
| Organic Chewable Base (Sugar SOY | 1.60 |
| FREE fermented Vitamin D3 900,000 IU | |
| Organic beet color | 6.80 |
| Total | 100.0 |

### Texture Analysis

### Example 7: Test Results-Average Tablet Hardness and Friability

Organic chewable tablets (with sugar, sugar free) as disclosed in examples 3A, 3B, 3C and 3D were tested for hardness along with non-granulated, non-organic tablets. The results are provided below in table 10.

TABLE 10
Average Tablet Hardness & Friability

| | Chewable Base | Chewable Base | Chewable Base | Chewable Base |
|---|---|---|---|---|
| | (Sugar Free) | (Sugar) | (Sugar Free) | (Sugar) |
| | (not granulated) | (not granulated) | (granulated) | (granulated) |
| No. of Tablets | commercial | commercial | Applicant's Sample | Applicant's Sample |
| Average. Tablet | 2.67 | 2.53 | 12.63 | 14.40 |
| Hardness (kp) | | | | |
| Friability (%) | 4.68 | 6.67 | 0.66 | 0.44 |

Commercial tablets are weak showing high amount of friability, and hence challenge to manufacture. Whereas, on the other hand, chewable tablets of the present disclosure showed USP friability below 1%, tablets retained hardness and are easily chewable.

### Example 8: Organic Fruit & Veg Fiber Tablet Characterization

Organic chewable vegetable and fruit fiber tablets were prepared using organic veg-fruit sweeteners. Tablet hardness for organic fruit fiber tablet, chewable sugar base, chewable sugar free base, and non-organic commercial product were analyzed, and the results are provided below in table 11.

TABLE 11
Average Tablet Weight & Hardness

| Organic Fruit & | Chewable | Chewable Base | Commercial Product |
|---|---|---|---|
| Veg Fiber tablet | Base (Sugar) | (Sugar Free) | (non-organic) |
| Average. Tablet | 2505 | 2499 | 2560 |
| Weight (mg) | | | |
| Hardness (kp) | 17.56 | 17.96 | 16.6 |

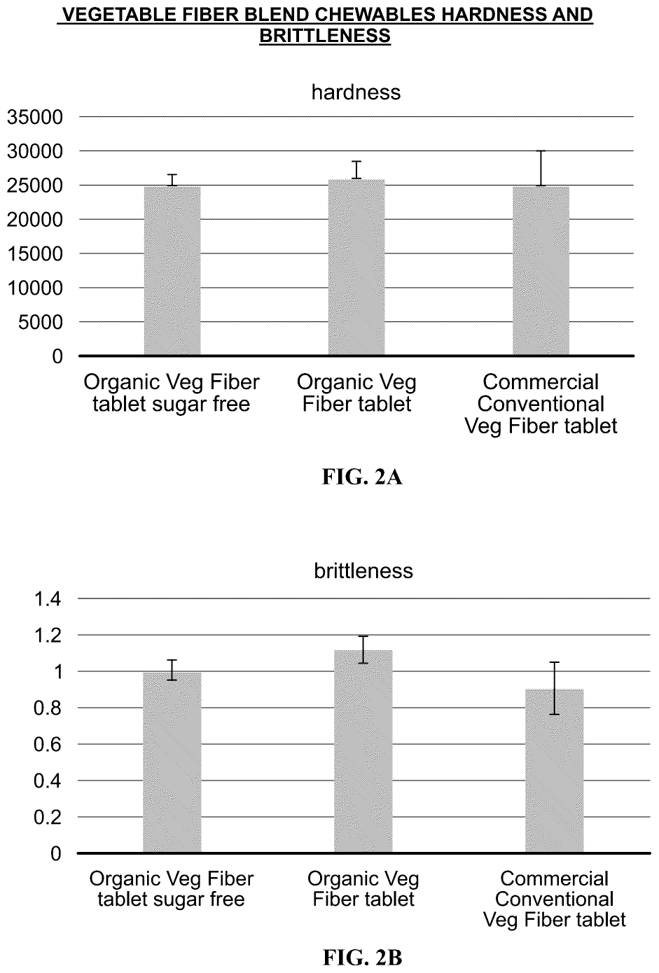

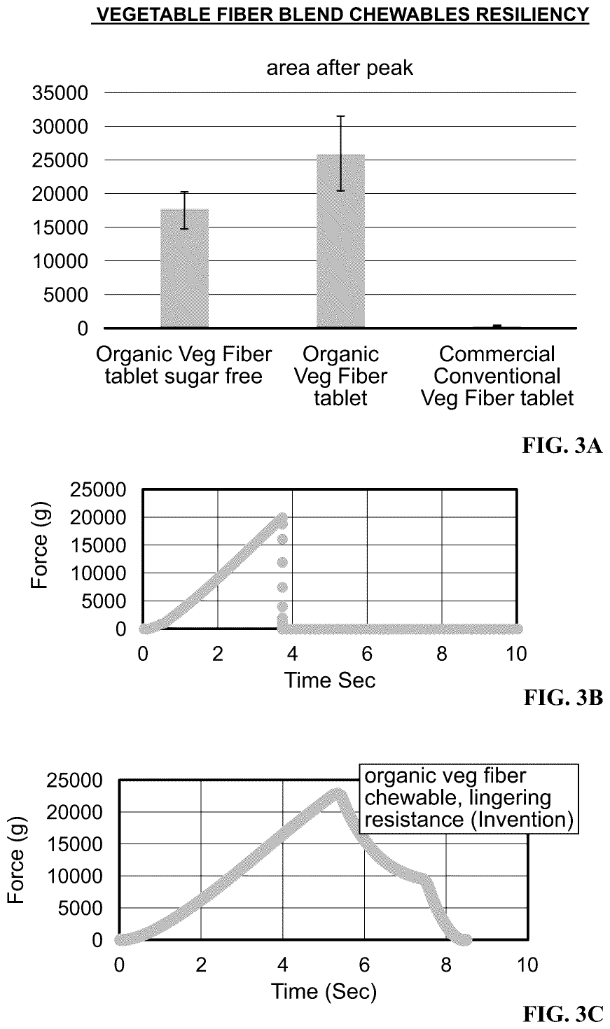

FIG. 2A shows hardness and FIG. 2B shows brittleness. FIGS. 2A and 2B show that tablet hardness and brittleness values were similar to those of the non-organic commercially available products while still being able to make a certified organic (>95%) product. This shows that present formulation was made using unique combination of ingredients and are able to meet or exceed synthetic and non-organic binders and fillers. FIG. 3A shows hardness values of Organic veg fiber tablet (sugar free), Organic Veg Fiber tablet against commercial conventional veg fiber tablet. FIG. 3B shows sharp break for conventional veg fiber whereas, FIG. 3C shows lingering resistance to break for present chewable Organic veg fiber tablets.

### Example 9: Organic Vitamin C Tablet Characterization

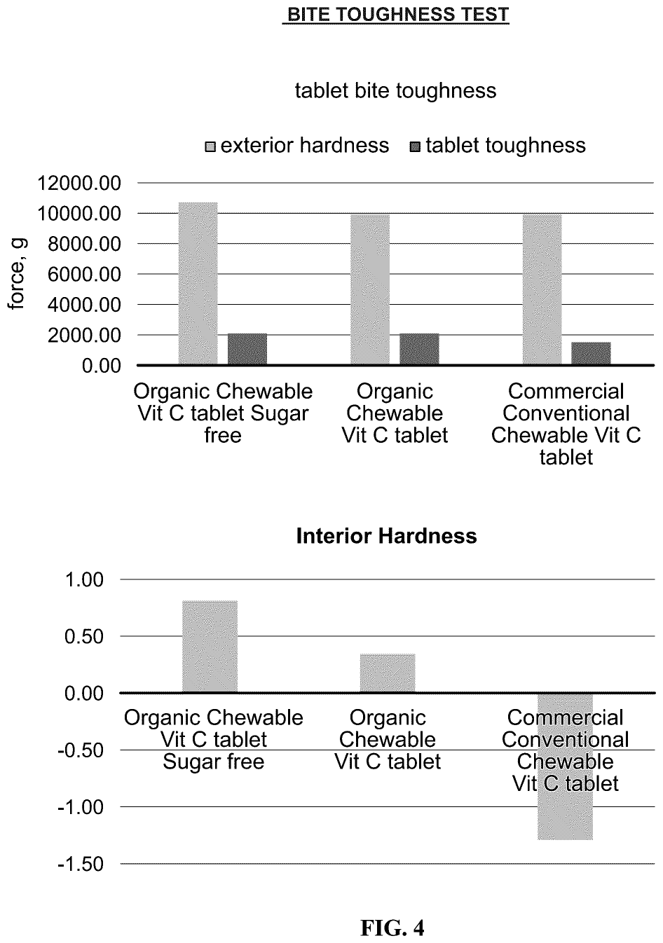

Chewable organic certified Vitamin C was subjected to texture profile analysis and physical parameters test results are provided in the table below and shown in FIGS. 4A & 4B.

TABLE 12
Organic Vitamin C

| Organic Vitamin C | Chewable | Chewable Base | Commercial Product |
|---|---|---|---|
| tablet | Base (Sugar) | (Sugar Free) | (nonorganic) |
| Average. Tablet | 2618 | 2630 | 2607 |
| Weight (mg) | | | |
| Hardness (kp) | 14.4 | 12.63 | 8.40 |
| Friability (%) | 0.49 | 0.66 | 0.29 |

FIGS. 4A and 4B show bite toughness and interior hardness test results of organic chewable Vitamin C tablets sugar free, organic chewable Vitamin C tablet in comparison to commercial chewable Vitamin C tablet. Organic chewable Vit C tablets are in range of commercial tablets, but interior hardness has more resistance/texture. Higher resistance correlates to chewiness in the core of the tablet.

### Example 10: Comparison Data—Sensory Evaluation Sugar Base

Sensory evaluation setup by a consultant was done on 41 participants between 2 locations on separate days. 4 samples were presented with random number 3-digit codes. 2 Samples each for Sugar and Sugar Free base, 2 samples for sugar and sugar free base with herbal active. Order of samples was varied by participant. Participants were asked to evaluate each sample individually before moving to next sample (water and cracker before starting and in between samples). Results are tabulated below:

TABLE 13
Sensory Evaluation Results

| Evaluation | Target | Criteria | Result |
|---|---|---|---|
| Tablet overall | >75% | Neither like nor | 82.0% |
| Like/Dislike | | dislike or better | |
| Texture | >50% | Just about right or better | 74.3% |
| Crumbliness | >50% | Just about right or better | 73.7% |
| Grittiness | <25% | Very gritty | 7.7% |
| Aftertaste | <25% | Somewhat unpleasant or lower | 17.9% |
| Aftertaste strength | <25% | Much too strong | 0% |

Results show minimal aftertaste effect, while few test subjects mentioned soft and chewy texture.

### Example 11: Sensory Evaluation of Sugar Free Organic Chewable Base

TABLE 14
Sensory Evaluation Results

| Evaluation | Target | Criteria | Result |
|---|---|---|---|
| Tablet overall | >75% | Neither like nor | 83.7% |
| Like/Dislike | | dislike or better | |
| Texture | >50% | Just about right or better | 85.3% |
| Crumbliness | >50% | Just about right or better | 76.3 |
| Grittiness | <25% | Very gritty | 2.6% |
| Aftertaste | <25% | Somewhat unpleasant or lower | 23.7 |
| Aftertaste strength | <25% | Much too strong | 10.5 |

Sugar and sugar free base comments were overwhelmingly positive on flavor, taste and sweetness. Foods that have lingering aftertastes typically have long sensation durations. Organic blend tablets (applicant's samples) had more resiliency and did not fracture and break like a conventional tablet, more confectionery-like. Results are shown in FIG. 3.

In another embodiment, the certified organic chewable tablets of the present disclosure provide numerous benefits such as (i) constitutes more than 95% certified organic components; (ii) better sensory characteristics such as crumbliness, tastiness, resiliency, unique texture, (iii) good tablet characteristics such as hardness, friability, chewiness, (iv) available in sugar free and sugar based chewable compositions and (v) xylitol free and allergan free.

While the compositions and methods of the disclosed and/or claimed inventive concept(s) have been described in terms of particular aspects, it will be apparent to those of ordinary skill in the art that variations may be applied to the compositions and/or methods and in the steps or in the sequence of steps of the method described herein without departing from the concept, spirit and scope of the disclosed and/or claimed inventive concept(s). All such similar substitutes and modifications apparent to those skilled in the art are deemed to be within the spirit, scope and concept of the disclosed and/or claimed inventive concept(s).

What is claimed is:

1. An organic chewable composition comprising:
(i) 0.1 to 95 wt. % of a chewable base composition comprising:
(a) 70 to 90.0 wt. % of at least one agglomerated organic primary sweetener blend;
(b) 1.0 to 10.0 wt. % of at least one organic lubricant and/or an organic binding agent;
(c) 1.0 to 10.0 wt. % of at least one co-processed organic secondary sweetener and an organic binding agent; and
(d) 0.1 to 10.0 wt. % of at least one additive; and
(ii) 0.01 to 5 wt. % of at least one functional ingredient.

2. The organic chewable composition according to claim 1, wherein the chewable composition is allergen free, xylitol free, more than 95% of the composition constitutes certified organic ingredients.

3. The organic chewable composition according to claim 1(i) (a), wherein the agglomerated organic primary sweetener is selected from the group consisting of organic stevia extract, organic thaumatin, organic tagatose, organic erythritol, organic monk fruit extract, organic cane sugar, organic raw honey, organic blackstrap molasses, organic real maple syrup, organic coconut sugar and blends thereof.

4. The organic chewable composition according to claim 1 (i) (a), wherein the organic primary sweetener is agglomerated blend of at least two organic sweeteners present in a ratio of 1:10 to 10:1.

5. The organic chewable composition according to claim 4, wherein the agglomerated primary sweetener blend is either (i) Organic Erythritol—Organic Tapioca Maltodextrin blend, (ii) Organic Monk Fruit—Organic Tapioca Maltodextrin blend, (iii) Organic Cane Sugar—Organic Erythritol blend, or (iv) Organic Erythritol—Organic Monk fruit.

6. The organic chewable composition according to claim 1(i) (b), wherein the lubricant is selected from the group consisting of an organic palm oil, organic coconut oil, organic stearic acid, organic carnauba wax, organic magnesium stearate and mixtures thereof; and binding agent is selected from the group consisting of food starch, guar gum, dextrose and organic gum acacia.

7. The organic chewable composition according to claim 1 (i) (c), wherein the secondary sweetener is selected from the group consisting of organic isomalto-oligosaccharides, organic chicory root extract, organic maize dextrin, organic rice syrup solids, organic corn maltodextrin, organic tapioca maltodextrin, organic rice maltodextrin, organic potato maltodextrin and blends thereof.

8. The organic chewable composition according to claim 1(i) (c), wherein the co-processed sweetener and binding agent is co-processed Tapioca Maltodextrin-Gum Acacia.

9. The organic chewable composition according to claim 1(i) (d), wherein the additive is selected from the group consisting of flavoring agents, coloring agents, lubricants, and glidants.

10. The organic chewable composition according to claim 9, wherein
(i) the flavoring agent is selected from the group consisting of orange, lemon, lime, blueberry, strawberry, mixed berry, cranberry, blackberry, cherry, apple, pineapple, pumpkin, pomegranate acai and mixtures thereof;
(ii) the coloring agent is an organic fruit, or an organic vegetable selected from the group consisting of orange, blueberry, strawberry, mixed berry, blackberry, cherry, apple, pineapple, pomegranate, pumpkin, acai, beet, cucumber, bilberry, and mixtures thereof;
(iii) the glidant is selected from the group consisting of an organic rice hull powder, silica and mixtures thereof; and
(iv) the lubricant is selected from the group consisting of an organic palm stearin, an organic palm oil, an organic coconut oil, an organic stearic acid, an organic carnauba wax, an organic magnesium stearate and mixtures thereof.

11. The organic chewable composition according to claim 1(ii), wherein the functional ingredient is a dietary supplement, a mineral, a vitamin, an enzyme, an over-the-counter drug or a pharmaceutical active.

12. The organic chewable composition according to claim 11, wherein the functional ingredient is an active ingredient selected from the group consisting of (i) organic Vitamin C derived from the natural ingredients from the group consisting of acerola cherry, Indian gooseberry, Elderberry, orange, lemon, and sweet lime; (ii) Veg-fruit fiber derived from natural ingredients selected from the group consisting of organic acerola, organic apple, organic beet, organic blackberry, organic banana, organic cranberry, organic carrot, cucumber powder, organic mango, organic pomegranate juice, organic pineapple, organic raspberry, organic strawberry, organic tart cherry, organic wild blubbery, organic white rice flour and mixtures thereof; (iii) Fermented Vitamin B12 and Vitamin D derived from natural ingredients selected from Organic Chewable Base (Sugar Free) [Organic Sweetener Blend Sugar-Free Blend: (Organic Erythritol, Organic Monk Fruit Powder), Organic Tapioca Maltodextrin, Organic Stearic Acid, Organic rice fiber powder, and Organic Gum Arabic], Organic Natural Flavors, Organic Beet Powder (color), and soy free fermented media consisting of Organic Rice Protein Powder, Organic Yeast (Saccharomyces cerevisiae), Organic Tapioca Maltodextrin, Organic Gum Arabic, Organic Black Strap Molasses, Probiotic Blend (Lactobacillus acidophilus, Lactobacillus rhamnoses and Bifidobacterium bifidum), Organic Safflower Oil, Bromelain and Papain. (iv) chewable multi-vitamin derived from natural ingredients selected from selected from Organic Chewable Base consisting of (Organic Sweetener Blend: (Organic Erythritol, Organic Cane Sugar), Organic Tapioca Maltodextrin, Organic Stearic Acid, organic rice fiber powder and Organic Gum Arabic), Organic Natural Flavor, Citric Acid and fermented media consisting of Organic Rice Protein Powder, Organic Yeast (Saccharomyces cerevisiae), Organic Tapioca Maltodextrin, Organic Gum Arabic, Organic Black Strap Molasses, Probiotic Blend (Lactobacillus acidophilus, Lactobacillus rhamnosus and Bifidobacterium bifidum), Organic Safflower Oil, Bromelain and Papain; and (v) Fermented Vitamin D derived from natural ingredients comprising Organic Sweetener Blend (Organic cane sugar, Organic Erythritol), Organic Tapioca Maltodextrin, Organic Stearic Acid, Organic Ground Rice hulls, Organic Natural Flavors, Organic Beet Powder (color), Organic Gum Arabica and soy free fermented consisting of Organic Rice Protein Powder, Organic Yeast (Saccharomyces cerevisiae), Organic Tapioca Maltodextrin, Organic Gum Arabic, Organic Black Strap Molasses, Probiotic Blend (Lactobacillus acidophilus, Lactobacillus rhamnoses and Bifidobacterium bifidum), Organic Safflower Oil, Cholecalciferol, Bromelain and Papain.

13. The organic chewable composition according to claim 1, wherein the composition is formulated into a tablet, a gummy or a candy.

14. An organic chewable tablet composition comprising:
(i) 0.1 to 95 wt. % of a chewable base composition comprising:
(a) 70 to 90.0 wt. % of at least one agglomerated organic primary sweetener blend;
(b) 1.0 to 10.0 wt. % of at least one organic lubricant and/or an organic binding agent;
(c) 1.0 to 10.0 wt. % of at least one co-processed organic secondary sweetener and an organic binding agent; and
(d) 0.1 to 10.0 wt. % of at least one additive; and
(ii) 0.01 to 5 wt. % of at least one functional ingredient.

15. The organic chewable tablet composition according to claim 14, wherein the chewable composition is allergan free, xylitol free and more than 95% of the composition constitutes certified organic ingredients.

16. The organic chewable tablet composition according to claim 14, wherein the chewable tablet composition exhibits
(i) average tablet hardness of about 8 to 30 Kiloponds (Kp),
(ii) friability below 1%, and
(iii) resiliency characterized by lingering resistance of 5 to 15 seconds.

17. The organic chewable tablet composition according to claim 14, wherein the composition exhibits chewability without fracture showing high bite toughness with exterior tablet hardness of about 10,000 Kiloponds (Kp), and interior tablet hardness of about 1.00 Kiloponds (Kp).

18. The organic chewable tablet composition according to claim 14, wherein the organic chewable tablet comprises:
(i) Vitamin C derived from the natural ingredients selected from the group consisting of acerola cherry, Indian gooseberry, Elderberry, orange, lemon, and sweet lime;
(ii) Veg fruit fiber derived from natural ingredients selected from the group consisting of organic acerola, organic apple, organic beet, organic blackberry, organic banana, organic cranberry, organic carrot, cucumber powder, organic mango, organic pomegranate juice, organic pineapple, organic raspberry, organic strawberry, organic tart cherry, organic wild blubbery, organic white rice flour and mixtures thereof;
(iii) fermented Vitamin B12 and Vitamin D derived from natural ingredients selected from the group consisting of Organic Chewable Base (Sugar Free) [Organic Sweetener Blend Sugar-Free Blend: (Organic Erythritol, Organic Monk Fruit Powder), Organic Tapioca Maltodextrin, Organic Stearic Acid, Organic rice fiber powder, and Organic Gum Arabic], Organic Natural Flavors, Organic Beet Powder (color), and soy free fermented media consisting of Organic Chewable Base (Sugar Free) [Organic Sweetener Blend Sugar-Free Blend: (Organic Erythritol, Organic Monk Fruit Powder), Organic Tapioca Maltodextrin, Organic Stearic Acid, Organic rice fiber powder, Organic Gum Arabic], Organic Natural Flavors, Organic Beet Powder (color);
(iv) chewable multi-vitamin derived from natural ingredients selected from selected from Organic Chewable Base (Organic Sweetener Blend: (Organic Erythritol, Organic Cane Sugar), Organic Tapioca Maltodextrin, Organic Stearic Acid, organic rice fiber powder and Organic Gum Arabic), Organic Natural Flavor, Citric Acid and fermented media consisting of Organic Rice Protein Powder, Organic Yeast (Saccharomyces cerevisiae), Organic Tapioca Maltodextrin, Organic Gum Arabic, Organic Black Strap Molasses, Probiotic Blend (Lactobacillus acidophilus, Lactobacillus rhamnosus and Bifidobacterium bifidum), Organic Safflower Oil, Bromelain and Papain; or
(v) fermented Vitamin D derived from natural ingredients selected from the group consisting of Organic Sweetener Blend (Organic cane sugar, Organic Erythritol), Organic Tapioca Maltodextrin, Organic Stearic Acid, Organic Ground Rice hulls, Organic Natural Flavors, Organic Beet Powder (color), Organic Gum Arabica and soy free fermented consisting of Organic Rice Protein Powder, Organic Yeast (Saccharomyces cerevisiae), Organic Tapioca Maltodextrin, Organic Gum Arabic, Organic Black Strap Molasses, Probiotic Blend (Lactobacillus acidophilus, Lactobacillus rhamnoses and Bifidobacterium bifidum), Organic Safflower Oil, Cholecalciferol, Bromelain and Papain.

19. A process for preparing an organic chewable base composition of claim 1, the process comprising:
(a) blending and micronizing 65 to 85 wt. % of at least two organic primary sweeteners using a mill through a 70 mesh to achieve the particle size of 100 to 210 μm;
(b) separately agglomerating 1.0 to 10 wt. % of aqueous solution of an organic secondary sweetener and spraying onto the sweetener blend obtained in step (a);
(c) separately milling 5.0 to 15.0 wt. % of an organic lubricant and 0.1 to 5 wt. % of organic binding agent through a 50-mesh to achieve the particle size of 150 to 300 μm and kept aside;
(d) separately preparing 5.0 to 15 wt. % of co-processed secondary sweetener and binding agent by mixing a secondary sweetener with an organic binding agent; and
(e) mixing the resultant of step (b), step (c) and step (d) along with 0.1 to 10 wt. % of an additive to obtain the organic chewable base.

20. The process according to claim 19 (a), wherein the agglomerated primary sweetener blend is selected from the group consisting of (i) Organic Erythritol—Organic Tapioca Maltodextrin blend, or (ii) Organic Monk Fruit—Organic Tapioca Maltodextrin blend, (iii) Organic cane sugar—Organic erythritol blend, and (iv) Organic Erythritol—Organic Monk fruit.

21. The process according to claim 19, wherein,
the organic secondary sweetener is organic tapioca maltodextrin,
the organic lubricant is organic palm fruit stearin,
the organic binding agent is organic gum acacia, and
the additive is silica.

22. An organic delivery system comprising:
(a) a chewable gummy or a chewable candy consisting of an organic chewable base composition of claim 1; and
(b) a functional ingredient.

23. The organic delivery system according to claim 22 (b), wherein the functional ingredient is a dietary supplement, a mineral, a vitamin, an enzyme, an over-the-counter drug or a pharmaceutical active.
